(19) United States
(12) Patent Application Publication
(10) Pub. No.: US 2015/0334994 A1
(43) Pub. Date: Nov. 26, 2015

(54) PET DATA MANAGEMENT AND INFORMATION STORAGE AND EXCHANGE SYSTEM

(71) Applicant: Chachi Prasad, Los Angeles, CA (US)

(72) Inventor: Chachi Prasad, Los Angeles, CA (US)

(21) Appl. No.: 14/721,921

(22) Filed: May 26, 2015

Related U.S. Application Data
(60) Provisional application No. 62/002,647, filed on May 23, 2014.

(51) Int. Cl. A01K 29/00; G08B 21/02; A01K 27/00

(52) U.S. Cl. CPC A01K 29/005; A01K 27/001; A01K 27/009; G08B 21/0269

(57) ABSTRACT

The present invention is directed to a specialized pet tag which integrates with a mobile software application developed for a smart phone or mobile device, such as an iPhone, ipad, Android, or Blackberry personal digital assistant, equipped with a camera, image sensor, RFID detector, or NFC sensor, that which facilitates data management and communication to a finder and/or the owner of a pet.

### CROSS REFERENCE TO RELATED PATENT APPLICATIONS

The present application is a Non-Provisional Patent Application which claims priority to U.S. Provisional Patent Application No. 62/002,647, filed May 23, 2014 the contents of which are incorporated by reference herein and which are a basis for a claim of priority.

### BACKGROUND OF THE INVENTION

1. Field of the Invention

The invention generally relates to the field of mobile applications for handheld devices, and more specifically to systems and methods for managing, organizing, identifying, creating data and communicating information to various parties about a pet.

2. Background Information

Pets are considered a member of the family to many people, and there are many aspects of their lives that mirror the needs of children especially when managing their care and comfort. Additionally, with their inability to speak and provide information related to their name, or owner, the animal is particularly vulnerable at times. For example, when a pet is lost or injured there is a sense of over-whelming need to help a defenseless animal unable to communicate with the rescuer or finder. The app also allows the pet owner to monitor where their pets is and their activity, for example monitoring the activities and whereabouts of a dog walker, or having the ability of the dog walker to scan the tag to update the owner as to their whereabouts and activity.

Pets often have data management needs very similar to humans, there is a need to document their address, location, activity levels, medical and insurance information, dietary concerns and even social calendar.

Previous pet products in the market tend to focus on only one aspect for pet data management. For instance when dealing with a lost pet, the previous attempts to rapidly identify pets so that owners could be alerted, such as the microchip were limited as to the type of information they provide, typically just veterinarian information or owner information which is often out of date. Additionally, the chip system requires a special reader typically only available in a vet office. Also, the chips are known to migrate within the animal and thus often go undetected even with the specialized reader.

Thus a need exists to create a more widely available pet identification and alert system that may rapidly identify and help render assistance in either finding the owner or providing the appropriate medical care to the lost pet.

The present application overcomes the limitations of pet identification devices and further adds dimensions of data management to ease the management of various aspects of pet ownership, identification. The primary focus of the pet data management system is the management of health records and pet insurance coverage and data, finding or locating lost pets, monitoring overall activity and a major social feature including a filter for pet photos. Also aggregating data to help owners find solutions to certain health problems and simple questions. Also having a 24 hour hotline for emergencies and the per's well-being.

### SUMMARY OF THE INVENTION

The embodiments of the present invention relate to a mobile application based system which facilitates data management and communication to a finder and/or the owner of a pet. Additionally, the mobile application system may be coordinated with a specialized tagging system to enhance the readability and amount of information stored and further allow for the connectivity to other pet owners, pet shelters, and vets.

One embodiment of the present invention is directed to a specialized pet tag which integrates with a mobile software application developed for a smart phone or mobile device, such as an iPhone, iPad, Android, or Blackberry personal digital assistant, equipped with a camera, image sensor, RFID detector, or NFC sensor, that enables a finder to verify or determine a variety of parameters related to the found pet. Some of the parameters include the identification of a lost pet, owner's information, the pet's medical needs and veterinarian information. Additional embodiments relate to a communication feedback system to the registered owner of the pet which allows the owners to receive communications as to any of a variety of parameters including the location of the pet and information related to the person who scanned the tag.

Further additional embodiments of the present invention are directed to a specialized pet tag which integrates with a mobile software application developed for a smart phone or mobile device, such as an iPhone, ipad, Android, or Blackberry personal digital assistant, equipped with a camera, image sensor, RFID detector, or NFC sensor, that facilitate data management and communication to a finder and/or the owner of a pet.

Such data management may include pet health records and other animal or personal data;

user groups, social media aspects, photo books and filters for altering photos; activity monitors; local parks and dog-friendly eateries based on GPS of animal tags. The app may further enable the owner the ability to filter out conversations about a certain ailment by breed or location, and/or have the ability to connect with others via breed, location, or via subject of conversation.

### BRIEF DESCRIPTION OF THE DRAWINGS

The principles of the present invention will be apparent with reference to the following drawings, in which like reference numerals denote like components:

FIG. 1 consists of a schematic flow diagram of the chain of communication created with an embodied tag and mobile application system.

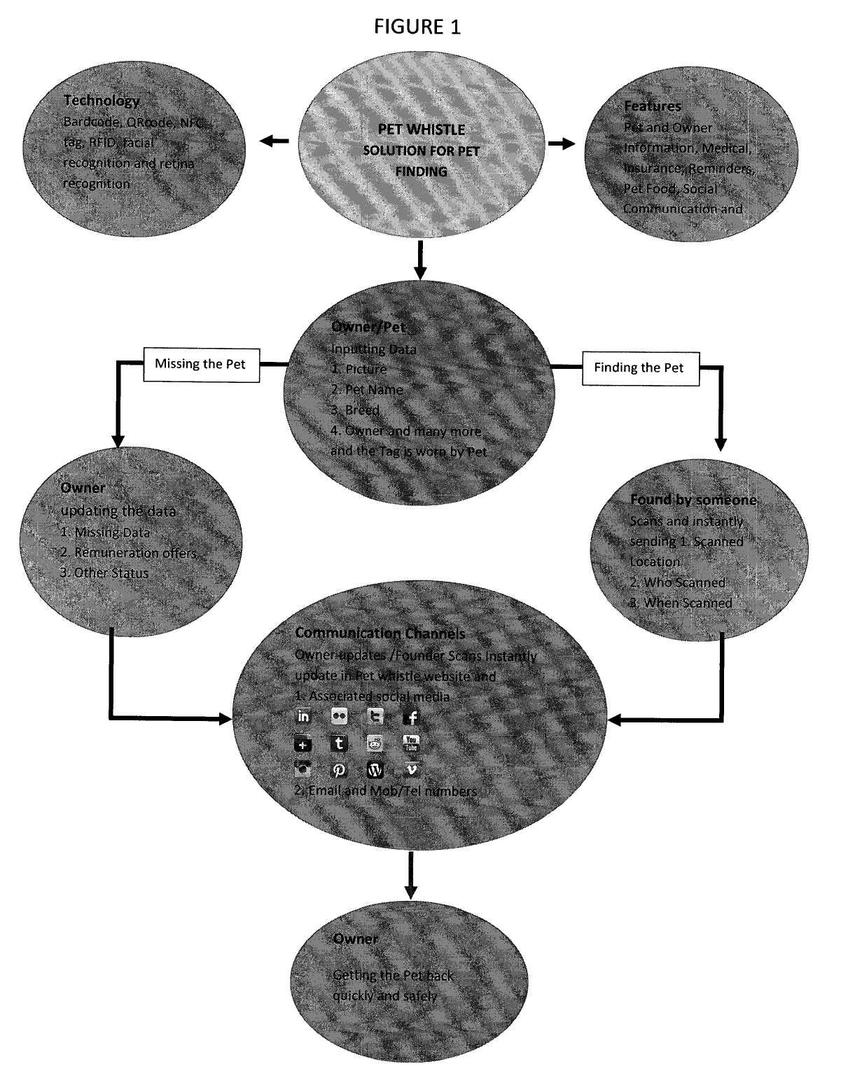

FIG. 2 shows a schematic flow diagram of the chain of communication created for a user Login or visitor Login.

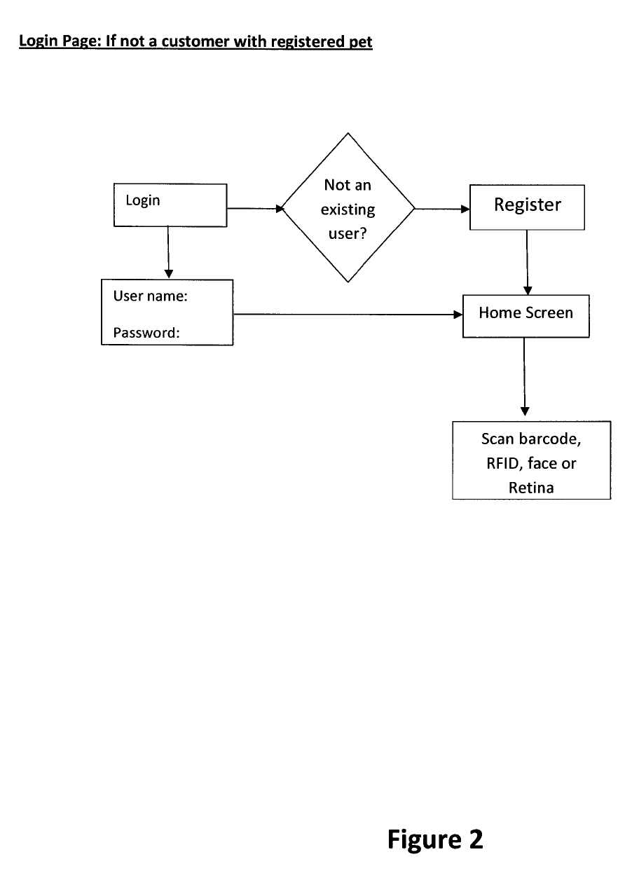

FIG. 3 shows an exemplary display screen of a Login or register screen on a mobile or computer device.

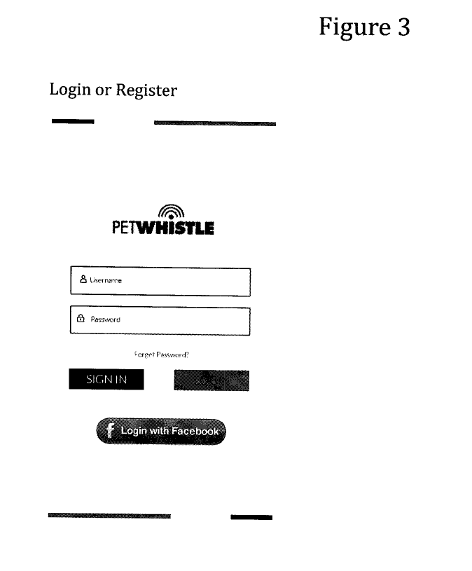

FIG. 4 shows a schematic flow diagram of the chain of communication created for the Home and Timeline screens for a user with a registered pet.

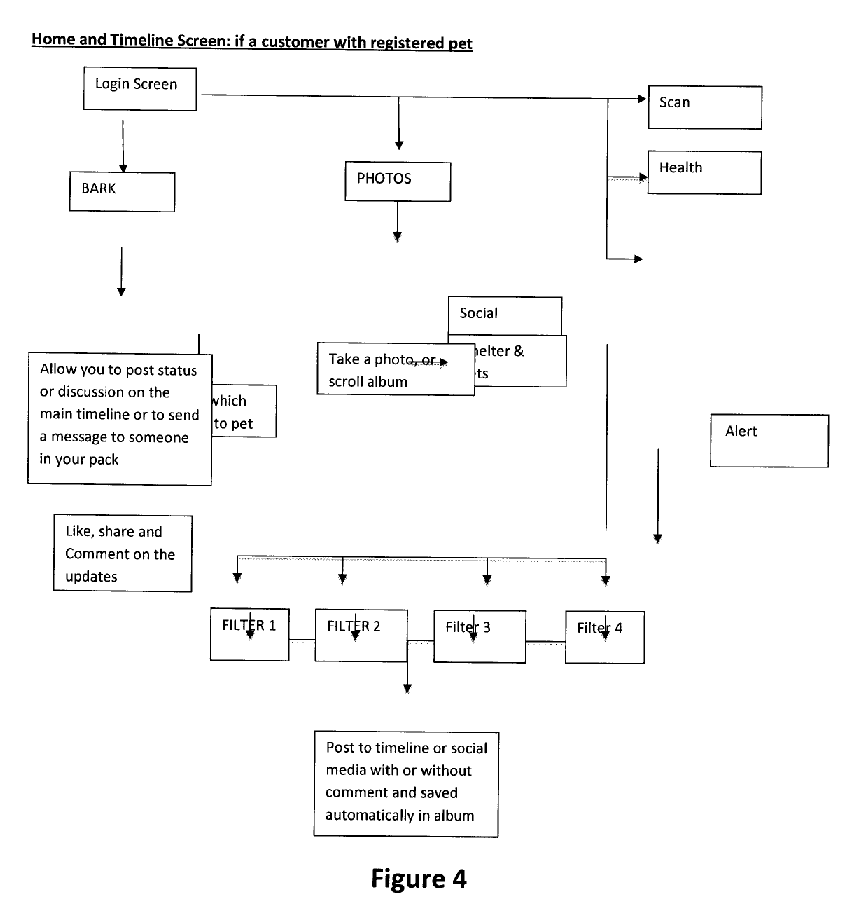

FIG. 5 shows an exemplary linked display screen for a Timeline entry screen on a mobile or computer device.

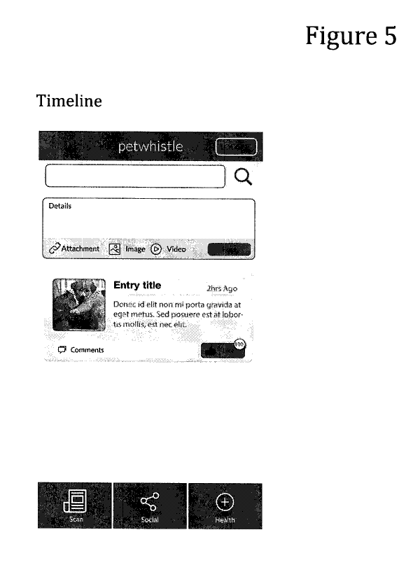

FIG. 6 shows a schematic flow diagram of the chain of communication created for the scanning of a pet ID.

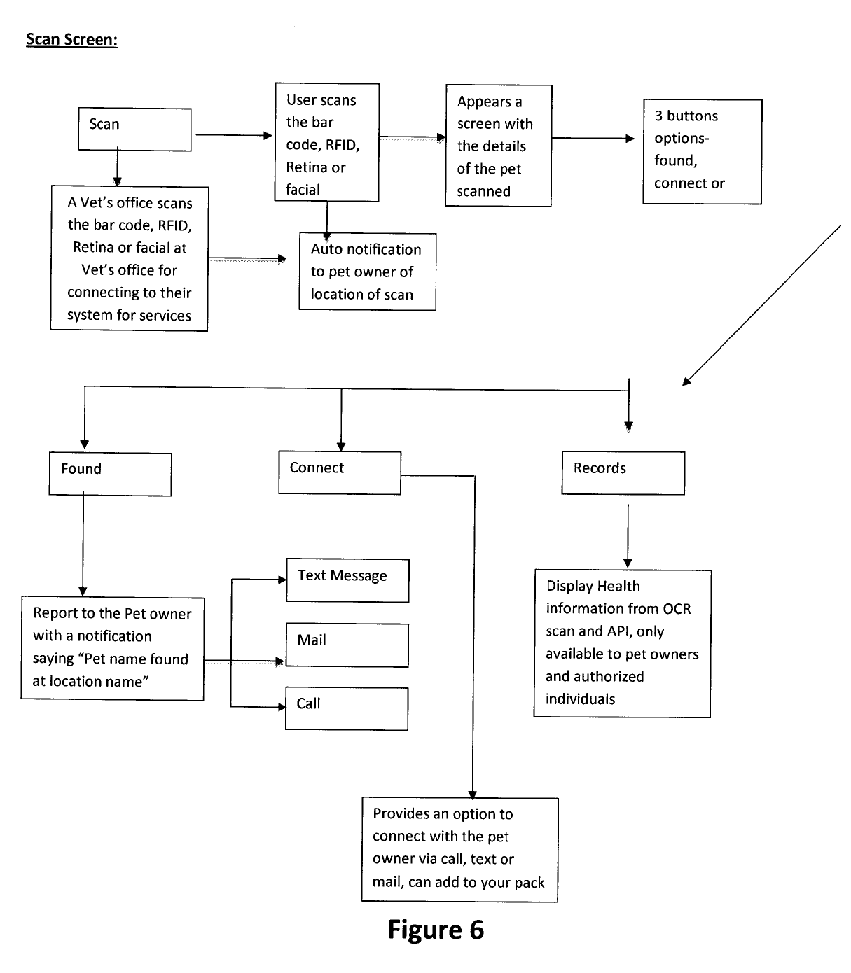

FIG. 7 shows an exemplary display screen showing the scanned animal information on a mobile or computer device.

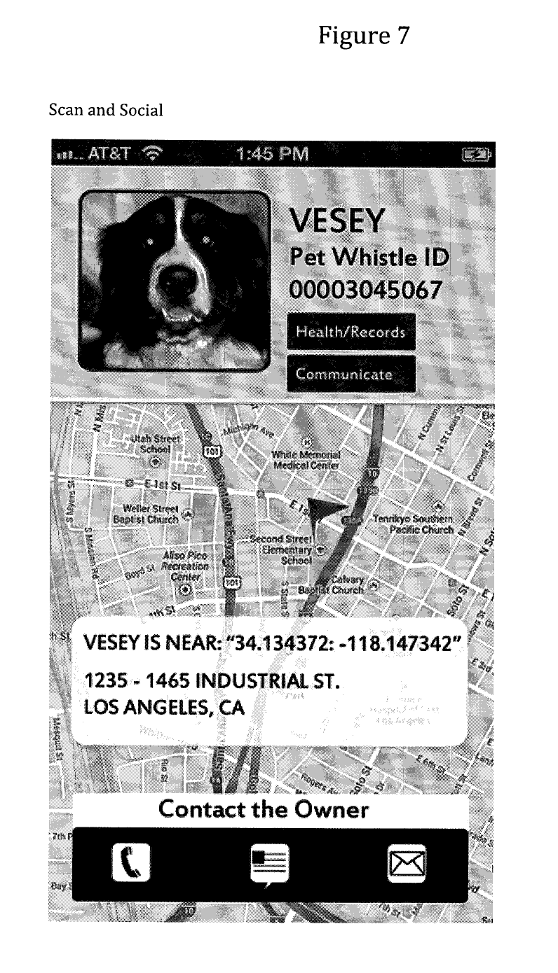

FIG. 8 shows a schematic flow diagram of the chain of communication created for the display of health information using API links and OCR technique.

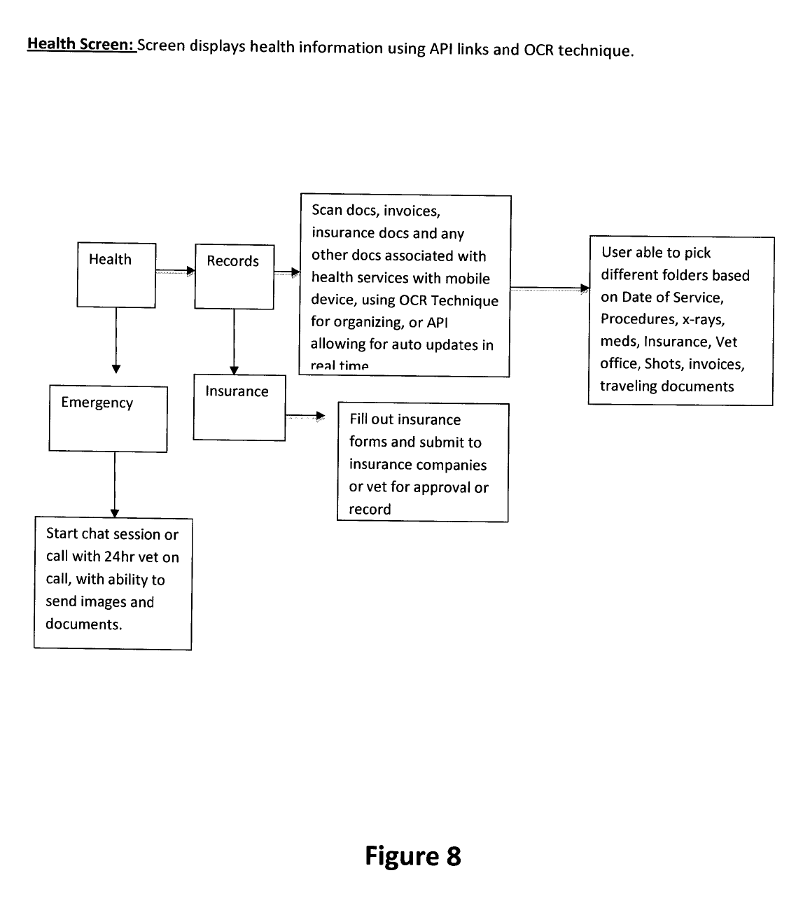

FIG. 9 shows a schematic flow diagram of the chain of communication created for the display of social media interactions.

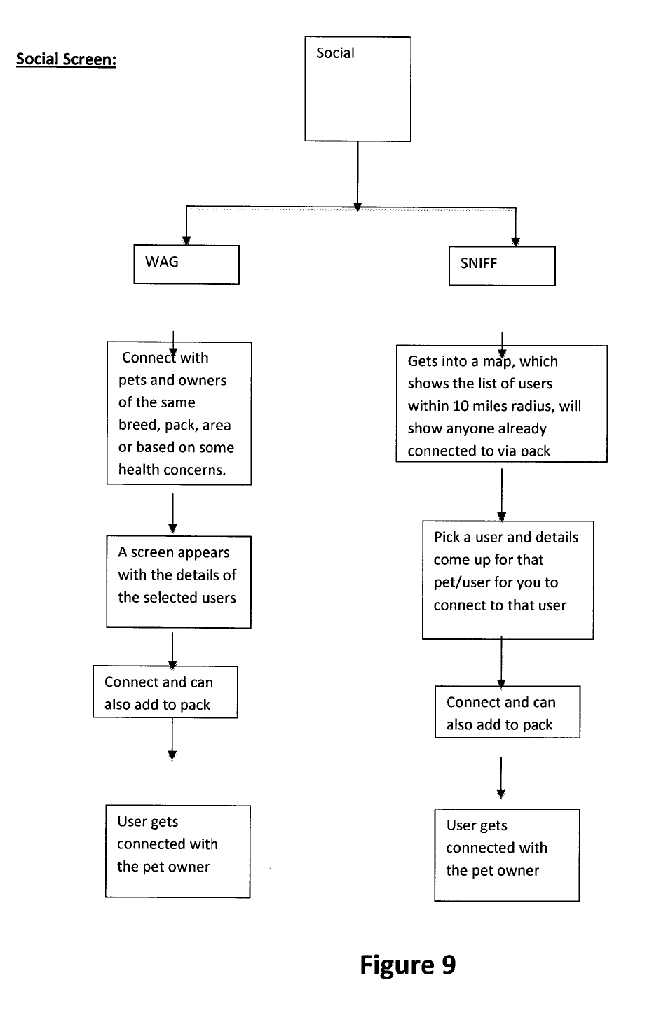

FIG. 10 shows an exemplary display screen showing the social photo album section information on a mobile or computer device.

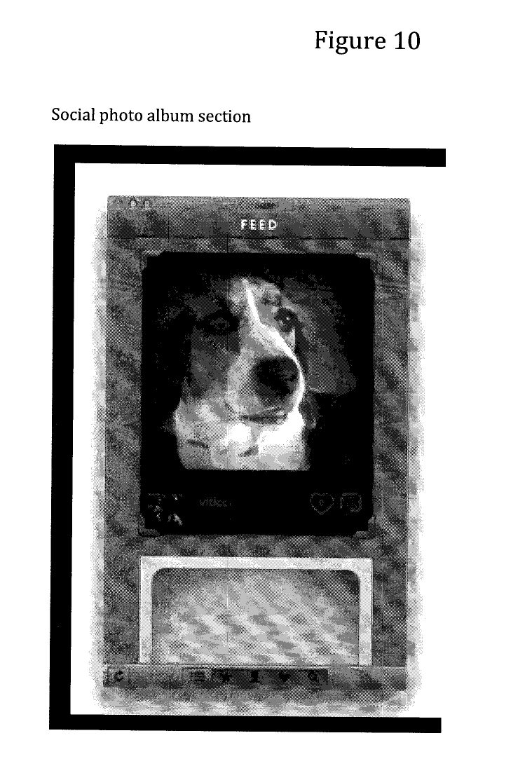

FIG. 11 shows a schematic flow diagram of the chain of communication created for the display of shelters and veterinarian offices.

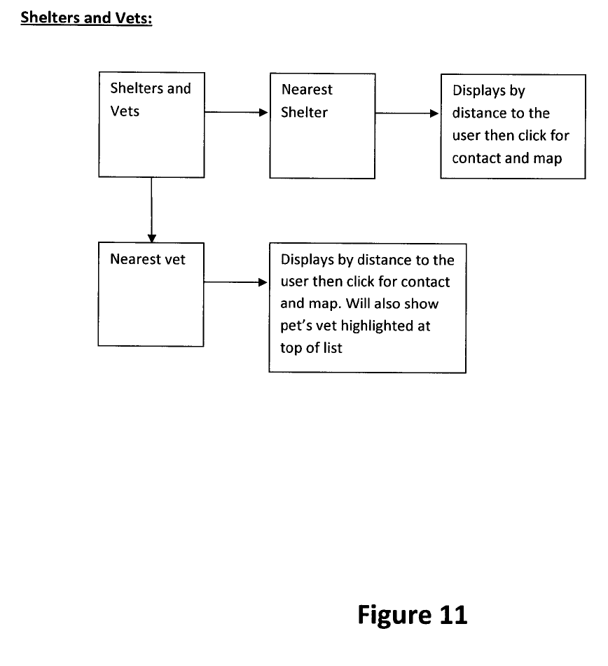

FIG. 12 shows a schematic flow diagram of the chain of communication created for the display of pet alert systems.

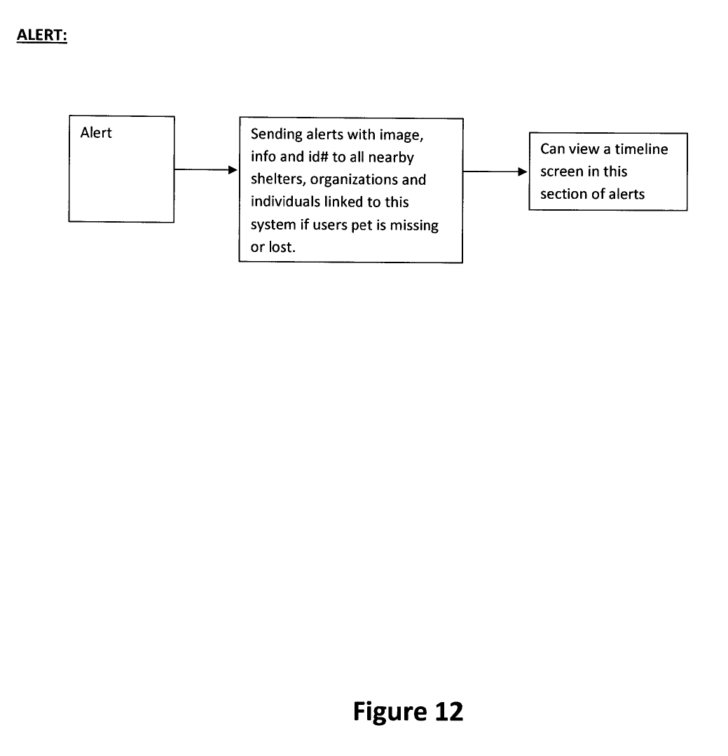

FIG. 13 shows an illustration of an embodied pet tag embedded in a color with the RFID information that is able to communicate with an App of the embodied invention.

FIG. 14 shows an illustration of another embodied pet tag embedded in a color with the RFID information that is able to communicate with an App of the embodied invention.

FIG. 15 shows another exemplary display screen showing the home page information for an embodied app as displayed on a mobile or computer device.

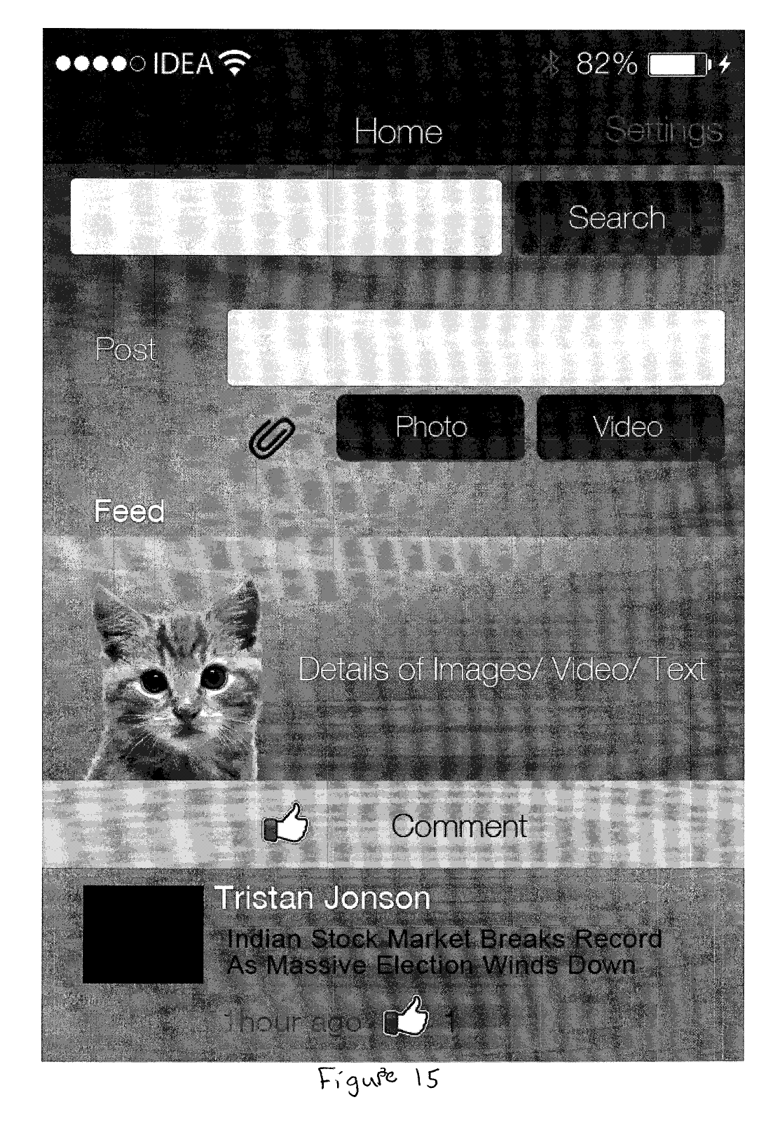

FIG. 16 shows another exemplary display screen showing the scan animal page information for an embodied app as displayed on a mobile or computer device.

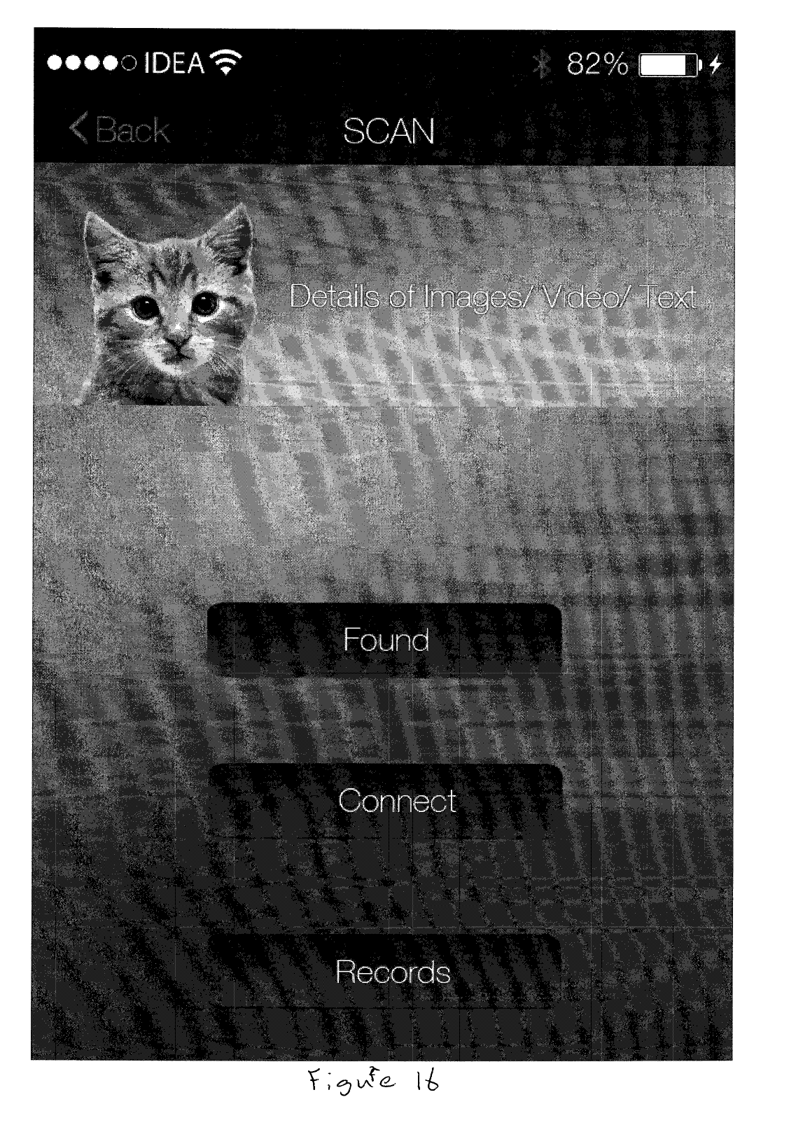

### DETAILED DESCRIPTION OF THE INVENTION

The present invention relates to a to a mobile application based system which facilitates data management and communication to a finder and/or the owner of a pet.

A first set of embodiments of the invention focus on locating and alerting pet owners if their pet is lost and found. A finder or temporary caretaker of said pet may scan their tag or collar which may comprise a specific barcode, a QRcode, a NFC tag, RFID, if the tag is lost or not available on the pet the software has a Facial recognition and/or retinal recognition feature to pull identifying data information.

The specialized tag is worn by the pet around its collar or harness and scanned with a mobile phone or the specific tag number can be manually put into the system via the web. The embodied mobile phone app serves as a Pet whistle so to speak, which alerts the owners that the pet has been found and scanned. Additional embodiments may allow for the pet owner to receive location information as to when and where the pet was scanned. Additional embodiments may include GPS tracking capabilities within the tag. Still further embodiments may provide identifying information related to the device and/or person scanning their pet. Additional information may be sent to the owner via social media sites.

An additional second set of Pet app aspects for the pet owner interface include:

Owner may store information in app and or tag including calendar and reminder option in the program for owners so they can be alerted of any meds and special dates for the pet, via mobile, including shots, flea med, grooming schedule and vet appointments.

Owners register the tag on line or via mobile inputting all the info regarding their pet and their info. They can expose some information to the public and some hidden for their security.

Owners can scan the tag once purchased and downloading a Pet app which will take them to the site for uploading their pet's and their owner's information.

Owners can go to a designated Pet app site and purchase a tag by signing up and the Pet app will initiate a tag and mail it to the owner with all their inputted info. They can also download the app and scan their pet's retina or face to be included in the database.

Owners are alerted by text or email of the location of the pet once a person scans the pet.

Owners are alerted who scanned the pet

Owners can send an alert via text, or email into the Pet app site and also on all social media sights and pet finder sights if the pet is lost. Information may also appear on the embodied app's timeline.

Owners can keep the name, breed, and sex of the pet. The date they purchased the pet and from which breeder, store or person.

Owners can keep track and be alerted of the pets diet and eating and going to the bathroom schedules

Owner has the option to belong to the Pet app board where people can discuss and share fun facts or serious matters regarding their pet

Owner can keep the pets medical records on the app.

Owner has the option to register the dog with local government that requires it, and keep that on file if asked to produce it to authorities they can produce it via mobile

Owner can keep AKC registration information on file as well.

This app will have the ability to forward special offers from pet food companies, stores, and manufacturers of pet supplies.

This app can instant message or send an email to the pet owner for any food recalls or pet supply recalls.

Pet app aspects related to the finder/scanner of an animal with or without an embodied collar include:

Finder has the ability to scan the tag on the pet from his or her smart phone after downloading a Pet app.

Finder has the ability to scan facial recognition and Retina to retrieve info on the pet from his or her smart phone after downloading a Pet app. The app will check the Facial recognition with back end data saved on a highly secure server that the owner inputs.

Finder has the ability to see the name, breed and sex of the pet.

Finder has the ability to see if the pet has any medical condition or needs meds or special care, handicaps or injuries.

Finder has the ability to contact the owner via phone call by choosing “call owner” button if the owner allows this to be done on the data input end.

Finder has the ability to email or text the owner “email” or “text” if owner has allowed this feature to be operable during set up.

Finder has the ability to post to social media sites that the dog is found by scanning tag or facial and retinal recognition.

Finder can alert the owner on their social media sites the owner sets up to be alerted on once the tag or pet is scanned via facial or retinal recognition.

Finder has the ability to find the nearest pet hospital or care center, including no kill shelters if needed.

Finder has the ability to contact the pet's vet or another party if designated by the owner

Finder has the ability to see the pet's eating and walking schedules if by chance they need to take care of the pet for a few days, until the owner can retrieve them.

Pet app aspects related to the embodied tag and/or collar identification system include:

A specialized tag in the form of a specific barcode, QRcode, NFC tag, and/or RFID is loaded with information related to the pet and/or its owners.

The tag is worn by the pet around its collar or harness and is meant to be scanned by a finder using an embodied Pet app via a mobile phone or other handheld device, or the tag number can be manually put in to the system via the web and a tag will be issued to the owner.

The tag can be made of any material, metal, plastic, silicone, leather, and worn around the collar as a charm.

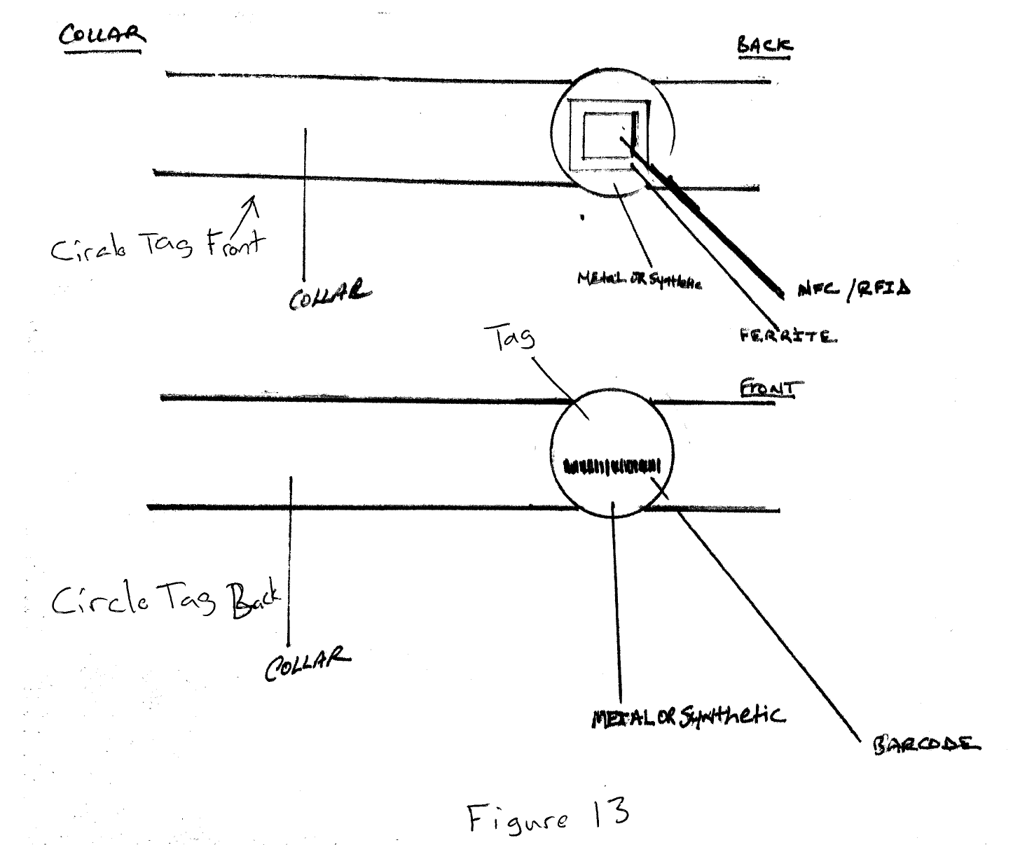

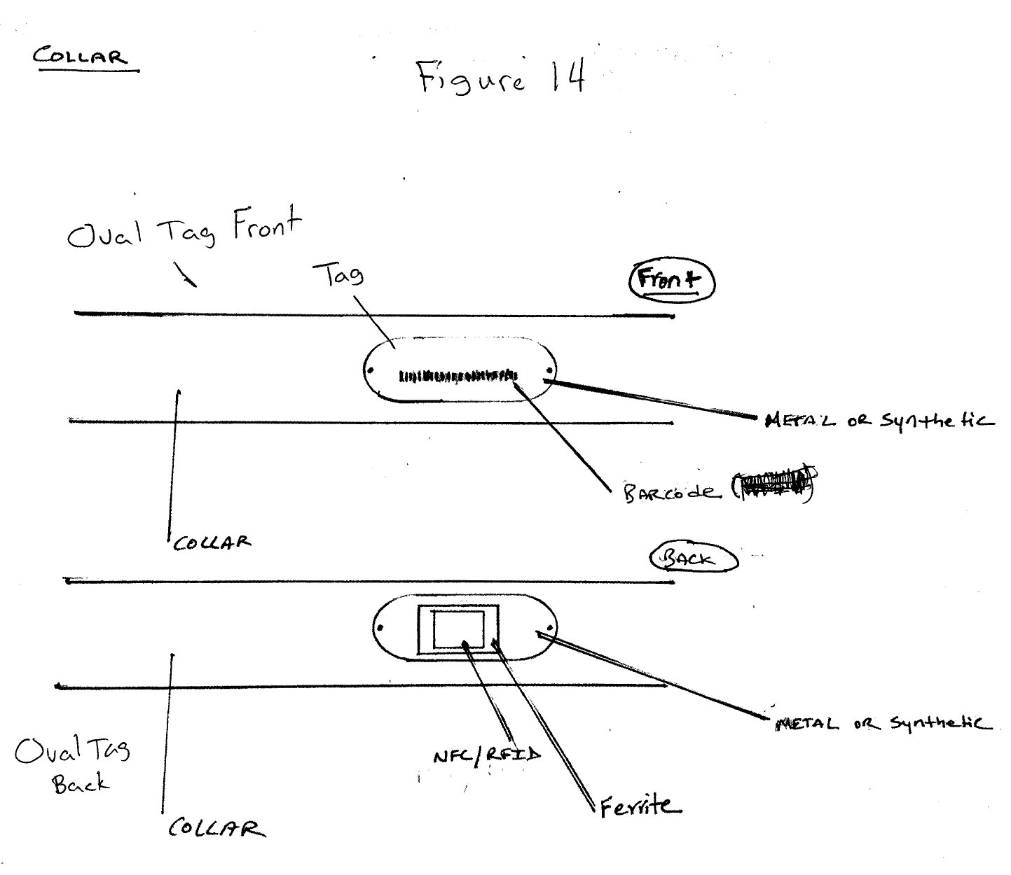

The code (such as in the form of an RFID) can be embedded in the collar it self As shown in example FIGS. 13 and 14.

The tag may further comprise GPS locator capabilities.

Further embodiments may utilize a barcode, RFID/NFC simultaneously to achieve communication and data management goals.

Additional embodiments include software for retina and facial recognition to enhance the features of the app.

Yet further apps and collars include GPS, and Bluetooth/Wifi signals to be sent to mobile phones.

Further General Capabilities of the Apps and/or Collars include:

The ability to scan via barcode, BLE, WIFI, NFC, RFID, Retina, and facial to locate a pet or health records

Further embodiments may include an automatic update to Health records via API or Scanning of docs via OCR.

Still further embodiments may include the ability to connect to other owners based on health, breed, location or for friendship to make play dates with pets.

Yet still further embodiments include the ability to add filters to pictures of pets. Such filters include characters like pirates, Charlie Chaplin look alike, FIREMEN, NURSE, DOCTOR, OR ADD clothing on the pets, such as a suit, pants coats, policemen, cowboys, zombies, super hero with cape scenery and enhancements to photos.

The ability to send location and an auto alert via notification to owner of pet once pet is scanned utilizing any or the above methodologies.

The ability to aggregate data on the timeline with ability to search via indexing of relevant data discussions.

The ability to chat with a 24 hr medical professional for emergencies

The ability to send alerts and broadcasts to shelters and organizations and social media that your pet is missing.

Embodiments include the ability to push or display a message (msg or txt) on a mobile device. (Similar to an Amber Alert type broadcast). Example, a pet is missing or lost, so the owner will activate a msg, which then can be broadcasted onto mobile devices via BLE or WIFI. The message can contain a picture of the pet along with a message indicating that it's lost, or needs meds, and a contact for them to call to return the pet.

Additionally the device on the collar can have a display, which can be programmed to have the pet's name shown, or a message that can be programmed by the owner, such as “Please help me I'm lost” “contact my owner Bob at 212-555-6767”.

The device may optionally have audio capabilities, where owner can program messages to be played or communicate via device. (A pet owner with a nanny cam can instruct the dog to get off the couch remotely).

The display can also show vitals for the pet's health such as heartbeat, breathing, temperature, and how much movement, or exercise the pet has had.

The ability for pets to be instantly checked in at a veterinary office when they walk in. Based on the scan of the collar which helps assist the vet office knows which animal they are working with.

The ability to be charged without taking out a wallet and receive invoices and receipts.

The ability to have a blinking light or indicator light on the device on collar

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### EXAMPLES RELATED TO PET LOCATION ASPECTS OF EMBODIED APP

### Example 1

### No Collar Tag System with Pet App Type Reader

In some contemplated embodiments of the present invention a person may find a lost and/or unattended animal and with the help of the Pet app type pet identification system they may identify the animal and find and alert the owner.

The user merely downloads the app, registers and opens the App and takes a picture of the animal and/or a retinal scan if available, and the information is then communicated and searched in either pet databases, or social media contexts and location parameters to identify the closest matches based on facial recognition identification or retinal scanning capabilities. A list of the most probable matches would come up based on facial recognition and location parameters (similar to a google type search). The facial recognition is kept in highly secure and encrypted database, and the app communicates with this database to ensure whom the pet belongs to. Another feature contemplated is a security feature for the owner to know when someone has scanned their pet, and the owner has the option to decide how much information to provide to the stranger/finder. An example to this would be in computer situations when allowing someone to enter into a personal computer remotely to assist the user is typically alerted as to who the person is trying to enter the computer and does the individual want to allow the person to enter.

### Example 2

### Specialized Tag Found Without Reader App

In some embodiments the animal may be found with the specialized tag but the finder may not have the capabilities of a smart-phone or scanning feature to retrieve information from the tag. In this case there will be instructions on the tag as to how the finder can manually input a code or information into a computer at a specific website to assist with the identification of the animal.

### Example 3

### Specialized Tag Found with Pet App Type Reader

In some embodiments the specialized tag may be found by a user who either has a Pet app application on their mobile device or is willing to download the app after seeing the lost pets tag.

The specialized tag on the found animal is scanned by a finder using a Pet app via a mobile phone or other handheld device, or the tag number can be manually put in to the system via the web and a tag will be issued to the owner.

Once the tag is scanned by the phone or placed into the system a system alert is sent to the owner of the location of the pet and the user who scanned the pet to their mobile and social media.

The person scanning will have the option to call, text or email the owner directly via pushing a button on the screen.

Also after scanning it will alert the person who found the pet if the pet requires any special needs, including meds, handicaps etc. and the veterinarian contact information. (An additional option is for the vet or a alternate individual or organization to be alerted as well as the owner, if the owners are out of town.)

The owner and finder can coordinate the return of the pet as rapidly as possible.

### Example of Everyday use of an Embodied Pet App:

The following example relates to the general use of the app for the user when a pet is not missing.

User LOGIN:

When the user first registers or logs in to the app, the user will see the following buttons and screen;

Scan—This feature allows people to scan the pet or tag. If it's a lost pet the user would press the “found” button which would prompt the user to report via email, text or call to the owner of the pet.

When the user is in a park and notices a dog that the user would like their pet to be buddies with or wish to add to their group or pack, the user can scan the pet or dog then press the “connect” button and it will send a message to the owner of that pet that the user wishes to connect with. Upon the pet owner accepting the invite the user will be connected.

When the user is at the Vet and the Vet scans the tag or pet, they can prepare the dog for its appointment and procedures. They can also see the medical and insurance records.

Health—this section is where all the records and health info are stored and retrieved. Once the user clicks on health the app will prompt the user to either go into records, insurance or emergency. Records, allows the user to store, organize and retrieve documents and information associated with vet visits, invoices and insurance in real time. The data management is accomplished in real time by building an API (application programming interface), which communicates with the veterinarian's software. So, whenever the Vet's office is updating any information on the users' pet, it is also getting updated in the users' health section of the app. The user can also upload docs and records via OCR (optical character recognition), which allows the user to scan the items via mobile device and store them in the appropriate folder or section. The can manage appointments for their pet for vets and grooming or any other appointments.

The emergency portion is for users to be able to connect with a live person/vet during emergency cases or injuries to a pet. This can be a chat session or a phone call.

Social—here the user can connect with people/pets on the app. The user can see and connect by type of breed or location. The user can connect with the same breed and send them a request to add to their pack or simply ask them a question regarding the breed. The user can make play dates for their dog based on their pack that they created i.e. group of pets that the user is most likely to hang out with or owners, or simply request to add them to the users group or pack.

Photos—feature where the user can take pictures of their pet and apply filters to the pictures, e.g. the user takes a picture of their pet then the app prompts the user to apply any of the multiple filters, such as a Charlie Chaplin lookalike, which would include a hat, short mustache, suit and a black and white background. The user can then post these images to social media, a timeline, or within an internal cache where the photos are saved in an album.

Bark—allows the user to post discussion or status on the main timeline and social media. It also allows the user to send a message to anyone in their pack or group.

Shelters & Vets—this allows anyone to find the nearest shelter and vets in accordance to their location or by a certain area. The user will be given a list based on their location or the area they have requested. This gives the ability to take the pet to the nearest shelter if the person cannot hold the pet for the owner, or if the pet has an injury that needs to be dealt with.

Alert—sends an Alert to all nearby shelters, organizations and social media that are linked to the system, similar to an “amber alert” type system but for lost or abducted pets. The message can come in the form of an email or push notification/message. This allows shelters to be aware of the missing pet if by chance someone drops it off. It will have all relevant data and image of the pet and a contact point for the owner.—It will also have a timeline of alerts being sent out by users.

Settings—where the user can add multiple pets to the app or toggle between the different pets if needed. Or update their profile, passwords, etc.

Home & Timeline (screen)—e.g. (like Facebook timeline or news feed) on the main screen where people are posting images and discussing items regarding their pets. The user can like, share or comment on the timeline. They can also search by relevant topics, i.e. golden retriever skin problem. The app will index and check all conversation related around this topic and display them for the user, who can comment and ask people questions around it.

Monitor Embodiments—include the ability to monitor and record pet activity throughout the day. An additional embodiment may include an activity monitor which records and may compile and store a pets activity throughout the day when animals are generally observed. The system may be similar to a “fitbit” type system for a pet that records activity and potential calories burned. Pets are typically unobserved for large parts of the day and they are unable to communicate certain information. By monitoring and recording their activity the owner is able to best monitor their nutritional caloric needs, as well as have the ability to detect an alert the owner to changes in activity that may be stressors to the pet. For instance if there is a rapid decrease in activity, this may be indicative of an illness or injury unobservable to the owner when the animal is in their presence. Animals often hide injuries or maintain a certain amount of stoicism when in the presence of their owner. Another exemplary feature would be one in which an owner sees a significant spike in activity at certain times, it may be indicative of a stressor that is occurring and can alert the owner. For example, if the data shows that every weekday at 3 pm the animal has very high activity it may be indicative that neighbors such as children are stressing the pet when they come home from school. Thus the system may allow the owner to identify a situation earlier and take remedial action if necessary.

Although the invention has been described with reference to the above examples, it will be understood that modifications and variations are encompassed within the spirit and scope of the invention. Accordingly, the invention is limited only by the following claims.

What is claimed is:

1. A mobile application system for assistance with the identification and recovery of lost pets; wherein said application receives input from the lost animal which assist with the identification of the animal; once the animal is identified or listed as to most probable identities; an alert system and/or additional information as to who the animal belongs to is provided to a finder; the pet finder is then able to help get the found animal either to their owner or to a place of treatment or safety.

2. A specialized pet tag which integrates with a mobile software application developed for a smart phone or mobile device, such as an iPhone, ipad, Android, or Blackberry personal digital assistant, equipped with a camera, image sensor, RFID detector, or NFC sensor, that facilitates data management and communication to a finder and/or the owner of a pet, as well as health records and other animal or personal data; user groups, social media aspects, photo books and filters for altering photos; activity monitors; local parks and dog-friendly eateries based on GPS of animal tags

3. A mobile application system comprising a mobile software application developed for a smart phone or mobile device, such as an iPhone, ipad, Android, or Blackberry personal digital assistant, equipped with a camera, image sensor, RFID detector, or NFC sensor, that facilitates data management and communication to a finder and/or the owner of a pet, as well as health records and other animal or personal data; user groups, social media aspects, photo books and filters for altering photos.

4. The system of claim 3 further comprising the ability to scan an animals information by a means selected from a barcode, BLE, WIFI, NFC, RFID, Retina, or facial recognition scan to locate a pet or to retrieve health records.

5. The system of claim 3 further comprising the ability to automatically update Health records via API or Scanning of docs via OCR.

6. The system of claim 3 further including the ability to connect to other owners based on health, breed, location or for friendship to make play dates with pets.

7. The system of claim 3 further including the ability to add filters to pictures of pets; wherein such filters include characters like pirates, Charlie Chaplin look alike, firemen, nurse, doctor, or add clothing on the pets, such as a suit, pants coats, policemen, cowboys, zombies, super hero with cape scenery and enhancements to photos.

8. The system of claim 3 further including the ability to send alerts and broadcasts to shelters and organizations and social media that your pet is missing.

9. The alert system of claim 8 further including the ability to push or display a message or text on a mobile device to multiple recipients; wherein, when a pet is missing or lost, the owner will activate a message, which then can be broadcasted onto mobile devices via BLE or WIFI.

10. The broadcast message of claim 9 wherein the message contains a picture of the pet along with a message indicating that it's lost, or needs meds, and a contact for them to call to return the pet.

11. The alert system of claim 8 wherein the device on the collar includes a display, programmed to have the pet's name shown, or a message programmed remotely by the owner.

12. The system of claim 3 wherein the device further includes audio capabilities, wherein an owner can program messages to be played or communicate via the device.

13. The system of claim 9 wherein the display shows vitals for the pet's health such as heartbeat, breathing, temperature, and how much movement, or exercise the pet has had.

14. The system of claim 3 wherein the device further includes the ability for pets to be instantly checked in at a veterinary office when they walk in.

15. The system of claim 14 wherein the device further includes the ability of the owner to be charged for veterinary office services without taking out a wallet and receive invoices and receipts.
